(19) United States
(12) Patent Application Publication
(10) Pub. No.: US 2015/0328183 A1
(43) Pub. Date: Nov. 19, 2015

(54) PHARMACEUTICAL COMPOSITION AND USE FOR APPLYING 7,7''-DIMETHOXYAGASTISFLAVONE IN INHIBITING TUMOR METASTASIS

(71) Applicant: NATIONAL CHIAO TUNG UNIVERSITY, Hsinchu (TW)

(72) Inventors: Kuang-Wen LIAO, Hsinchu City (TW); Ching-Min LIN, New Taipei City (TW); Yu-Ling LIN, Tainan City (TW)

(21) Appl. No.: 14/713,813

(22) Filed: May 15, 2015

(30) Foreign Application Priority Data
May 19, 2014 (TW) 103117534

(51) Int. Cl. A61K 31/352

(52) U.S. Cl. CPC A61K 31/352

(57) ABSTRACT

A pharmaceutical composition for inhibiting tumor metastasis is disclosed. The pharmaceutical composition includes a therapeutically effective amount of 7,7″-Dimethoxyagastisflavone (DMGF) and a pharmaceutically acceptable carrier. The present invention also discloses a use of DMGF for manufacturing a pharmaceutical composition applied to inhibit tumor metastasis. The application of the pharmaceutical composition and the use of the present invention are advantageous for inhibiting tumor metastasis efficiently.

### CROSS REFERENCE TO RELATED APPLICATIONS

This Non-provisional application claims priority under 35 U.S.C. §119(a) on Patent Application No(s). 103117534 filed in Taiwan, Republic of China on May 19, 2014, the entire contents of which are hereby incorporated by reference.

### BACKGROUND OF THE INVENTION

1. Field of Invention

The present invention relates to a pharmaceutical composition and use of a substance applying for making the pharmaceutical composition.

2. Related Art

Polyphenolic compound is commonly existed in daily diet and herbal ingredients, such as vegetables, fruits, beans, wine, tea, and gingko all contain different polyphenolic compounds which provides different biological activities, e.g. anti-inflammatory, anti-cancer, or anti-aging efficacy, to organisms. In particular, paclitaxel (Product Name: Taxol) extracted from bark of Taxus brevifolia Nutt. is successfully demonstrated to have the potential of cancer treatment, thus drawing larger attention from the medical field. Hence, the biological activity of other substances extracted from Paclitaxel is very worthy for study and development.

The substances extracted from branches and leaves of Taxus brevifolia Nutt. include biflavonoid compound other than Paclitaxel. Study has been shown that the biflavonoid compound includes broad biological activities, e.g. of anti-fungal, anti-inflammatory, anti-viral, anti-oxidation or anti-tumor efficacy. However, different biflavonoid compound possesses different biological activities. Even different biflavonoid compounds has anti-cancer potential, its mechanism and application are still mostly unknown.

7,7″-Dimethoxyagastisflavone (DMGF) is one of the biflavonoid compounds. However, few studies of mechanism and efficacy of DMGF are available. Only the ability of inhibiting Aspergillus flavus secreting aflatoxin is found, and the subsequent studies are failed to have further breakthrough. Since different biflavonoid compounds have different biological activities, it is an important subject in pharmaceutical industry to observe whether DMGF comprises the effect of inhibiting cancer, as paclitaxel does.

### SUMMARY OF THE INVENTION

Given the lack of prior art, the inventor provides the present invention after research and development. The purpose of the present invention is to provide a biflavonoid compound pharmaceutical compositions and a use of biflavonoid compound for preparing pharmaceutical compositions, which is particularly using 7,7″-Dimethoxyagastisflavone to provide the effect of inhibiting tumor metastasis.

In detail, the present invention provides a pharmaceutical composition for inhibiting tumor metastasis which comprises 7,7″-Dimethoxyagastisflavone(DMGF, hereinafter referred to DMGF) and a pharmaceutically acceptable carrier, excipient or a combination thereof.

The present invention further provides use of a DMGF applied for preparing a pharmaceutical composition used for inhibiting tumor metastasis.

To clearly present the technical features of the present invention, specific terms are defined first, and the present invention is further illustrated. The term “tumor metastasis” refers to the transferring process of the tumor cells from primary site to other site away from the primary site to grow continuously; in other words, it means the process of tumor cell spreading to other tissue or organ. The process is similar to cell migration, but in the cell migration process, tumor cells have to penetrate extracellular matrix (ECM) or basement membrane to invade into other tissues or organs, which will be illustrated later. Generally speaking, benign tumor does not perform metastasis; hence, “metastasis” is also called malignant metastasis. Otherwise, the tumor cells mentioned in the present invention indicates malignant tumor cells.

Tumor cells need to spread to other body sites by crossing or bypassing adjacent cells, and entering into circulatory system. Furthermore, tumor cells have to migrate by recombine cytoskeleton or adhere to other cells or extracellular matrix.

“Cell migration”, or so called “cell movement”, must change its position through cytoskeleton recombination. Cytoskeleton includes microfilament, microtubule, or intermediate filament. Microfilament is a fibrous structure composed of actin, which containing monomeric actin monomers having binding site of adenosine triphosphate (ATP) on the surface. While actin monomers combine with adenosine triphosphate, they can be connected to one polymer actin chain which is called fibrous actin (F-Actin). Two fibrous actins twist with each other to form a microfilament helix. Microfilament is also able to combine with microtubule, intermediate filament and other protein molecule for cell migration or movement. Therefore, inhibiting the polymerization of fibrous actin helps to inhibit tumor metastasis. The term “polymerization of fibrous actin” means that the fibrous actins twist to each other to form a microfilament helix.

In addition, the term “Extracellular matrix” means the macromolecules synthesized in cell and secreted to the extracellular to distribute on the surface of or between the cells. These macromolecules are mostly composed of polysaccharide and protein or proteoglycan, e.g. elastin or fibrin. After tumor cells adhere to the surface of plasma membrane or protein of extracellular matrix, they further migrate through adjacent cells with recombinant cytoskeleton, until encountering obstacles hard to bypass. Obstacles are usually basal lamina or basement membrane surrounding tissue. Tumor cells may synthesize and secrete a variety of protease, and degrading the proteins of extracellular matrix and basement membrane, thus entering into the circulatory system, e.g. entering into blood vessel. Generally speaking, the processes of tumor cells adhering to the protein of cell membrane surface or extracellular matrix, secreting various kinds of protease to degrade the protein of extracellular matrix and penetrating the extracellular matrix, are able to be classified into the process of “tumor metastasis”.

Tumor cells need to leave the circulatory system by degrading the extracellular matrix to penetrate the wall of blood vessel or lymphatic vessel. Hence, inhibiting the ability of tumor cells degrading extracellular matrix, and inhibiting the expression of protease of tumor cells may further inhibit tumor metastasis. The term “expression of protease” means the ability to synthesize protease of RNA level or protein level.

Tumor cell metastasis ability is affected by its extracellular matrix and basement membrane degradation ability, and also by the protease synthesis and secretion ability of tumor cells. The protease synthesized or secreted by tumor cells includes matrix metalloproteases (MMP). Matrix metalloproteases, including the collagen and extracellular matrix degradation ability, was discovered in 1962. At least 28 different kinds of matrix metalloproteases have been found. Matrix metalloproteases-2 (MMP2) belonging to gelatinase also has the ability to degrade extracellular matrix. The matrix metalloprotease is also considered to have high correlation degree with tumor metastasis and angiogenesis. Matrix metalloprotease helps cancer cells spread out by assisting angiogenesis and decomposing tissues. Hence, inhibiting the expression of matrix metalloproteases-2 may inhibit the ability of degrading extracellular matrix, further inhibiting tumor metastasis. Otherwise, the term “expression of matrix metalloproteases-2” means the ability to synthesize matrix metalloproteases-2 of RNA level or protein level.

In addition, the term “inflammatory response” means defense mechanism generated by several immune system or immune cells in human body in order to against foreign or endogenous stimuli. Pro-inflammatory factor manipulated mainly by nuclear factor-κB (NF-κB), e.g. tumor necrosis factor α (TNF-α) is highly correlated with the inflammatory response. Otherwise, inflammatory response can be divided into short-term acute inflammatory and long-term chronic inflammation. Although short-term acute inflammatory has been proved have the efficacy of repairing and rebuilding damaged tissue, excessive and long-term chronic inflammation may cause chronic disease such as cancer, diabetes, cardiovascular disease etc. Especially in recent years, many of the molecular, cells, or immunological researches have directed that inflammatory response is closely related to cancer, e.g. promoting angiogenesis surrounding tumor cells and several pathological effects such as invasion or metastasis of tumor cells. Therefore, inhibiting the secretion of tumor necrosis factor α from immune system may inhibit inflammatory response, further inhibiting tumor metastasis.

The “7,7″-Dimethoxyagastisflavone” applied in the present invention, or briefly called “DMGF” not only indicates to DMGF, but also includes derivatives generated by basic DMGF structures which are chemically or biologically modified or substituted. The derivatives contain the same property as the basic DMGF structure or similar property with the DMGF structure.

The term “effective amount” means the dose of DMGF capable of inhibiting tumor metastasis. In the present invention, the dose of DMGF preferably ranges from 0.8 to 4 mg per kilogram and per day. Otherwise, tumors may, for example, but not limited to be melanoma, lung cancer or colorectal cancer, and the tumor metastasis can be suppressed by the component of biflavonoid compound DMGF.

As mentioned above, the pharmaceutical composition and use for manufacturing the same. It applies biflavonoid compound DMGF which provides apparent efficacy on inhibiting tumor cells by different pathway.

### BRIEF DESCRIPTION OF THE DRAWINGS

The present invention will become more fully understood from the subsequent detailed description and accompanying drawings, which are given by way of illustration only, and thus are not limitative of the present invention and wherein:

FIG. 1 shows data figure for calculating the B16F10 cells migrating through transwell plate to the lower well in example 2.

FIG. 2 shows data figure of MMP2 expression level in example 3.

FIG. 3 shows the result of fibrous actin polymerization of SVEC4-10 in example 4.

FIG. 4 shows the result of the tumor volume of each mouse in example 5.

FIG. 5 shows the result of TNF-α secreted by mouse splenocyte in example 6.

### DETAILED DESCRIPTION OF THE INVENTION

The present invention will be apparent from the following detailed description, which proceeds with reference to the accompanying drawings, wherein the same references relate to the same elements.

In one embodiment of the pharmaceutical composition inhibiting tumor metastasis of the present invention, the pharmaceutical composition is prepared through biflavonoid compound 7,7″-Dimethoxyagastisflavone (hereinafter referred to DMGF) with therapeutically effective amount and a therapeutically acceptable carrier. DMGF is extracted from dried leaf of Taxus media var. Hicksii, preferably extracted with High Performance Liquid Chromatography (HPLC) to obtain extracts containing DMGF with more than 98% purity. HPLC is well-understood by the person having ordinary skill in the art, and is not repeated here.

In addition, the pharmaceutical composition of the present invention can be administered to patients orally with any orally acceptable dosage form, for example but not limited to capsules, drug tablets, aqueous suspensions or solutions. With respect to drug tablets, the most commonly used carriers include lactose and corn starch. Otherwise, lubricants may be additionally used, such as magnesium stearate. Regarding capsule form of oral administration, diluents include lactose and dried corn starch. While using aqueous suspensions for oral administration, the active ingredient DMGF, emulsifier and suspending agent are combined together. In other embodiment, specific sweeteners, flavoring agents and colorants may be added for easily administering to patients.

The pharmaceutical composition of present invention may be administered by rectal administration with suppository dosage form. The pharmaceutical composition of the present invention further includes suitable and non-irritating excipients mixture for manufacturing suppository dosage. The excipients mentioned above are solid form at room temperature, but changing into liquid form at rectal temperature, thus dissolving in the rectum and releasing active ingredient DMGF. Specifically speaking, excipients include coconut butter, beeswax and polyethylene glycols. In addition, pharmaceutical composition can be deployed to become ointment dosage form for skin topical administration. Carriers in the ointment dosage form capable of topical administration includes but not limited to mineral oil, liquid petrolatum, propylene glycol, polyoxyethylene, polyoxypropylene compound, emulsifying wax, or water. In addition to the above dosage forms, the pharmaceutical composition can also be prepared in the injection form, which is not a limitation of the present invention.

Preferably, acceptable DMGF dosage form for patients with tumors ranges from 0.8 to 4 mg/kg per day. “Patients acceptable dosage range” means the DMGF dosage range contained in the pharmaceutical composition. The DMGF dosage range contained in the pharmaceutical composition can be adjusted with administration route, the subject or its physical condition. Generally speaking, oral forms of the pharmaceutical compositions require higher doses for conveniently administration. In practical use, formulations of pharmaceutical compositions may be powder dosage, and each powder dosage comprises pharmaceutical composition with one dosage unit. In other embodiment, one dosage unit may be composed of pharmaceutical composition dispersed in several sub-dosages units or packages, for example, dispersed in 2 to 3 tablets or capsules packed in one blister pack.

To be more precisely, after administering the pharmaceutical composition of the present invention to patients with tumor, DMGF inhibits the ability of tumor cells degrading extracellular matrix by inhibiting the expression of matrix metalloproteases-2. DMGF may further inhibit tumor metastasis by inhibiting the expression of protease of tumor cells, the polymerization of fibrous actin, or the secretion of tumor necrosis factor a from immune cells. Preferably, DMGF may inhibit melanoma, lung cancer or colorectal cancer metastasis, further indirectly eliminating, inhibiting, improving, alleviating, or preventing cancer or its symptoms, or delaying, stopping, reversing tumor proliferation rate, or achieving medical effects similar to the above mentioned purposes.

The present invention further provides a use of a biflavonoid compound DMGF applied for manufacturing pharmaceutical composition used for inhibiting tumor metastasis. However, the pharmaceutical composition is the same or similar to that described corresponding to prior embodiment, and thus will not be described here.

As mentioned above, the pharmaceutical composition and use of the present invention for applying DMGF in inhibiting tumor metastasis provides significant efficacy.

### EXAMPLE 1

### Preparing DMGF

DMGF used in the present invention was obtained from extracts including DMGF by high performance liquid chromatography on a Waters HPLC system (Waters 2796 Bio-separation Module) equipped with C18™ Symmetry column (250 mm 4.6 mm i.d.) with a particle size of 5mm. Isocratic elution was monitored at 254 nm under room temperature and performed for methanol and water (30:70, v/v at a flow rate of 0.5 mL/min), thus obtaining the extracts containing DMGF. 5 mg extracts weighed under room temperature is considered to be one unit dosage which was packed into powders or flat capsules dosage form and preserved under room temperature.

### EXAMPLE 2

### Assay for Metastasis Inhibition by DMGF

B16F10 mouse melanoma cells were maintained in 1% antibiotics of Dulbecco's modified Eagle's medium (DMEM; Gibco/Invitrogen, Carlsbad, Calif., USA) supplemented with heat-inactivated 10% fetal bovine serum (Gibco/Invitrogen, Carlsbad, Calif., USA). Antibiotic were preferably penicillin/streptomycin/amphotericin. Cell suspension (3×10⁴) were treated with or without 1 μg/ml DMGF in the upper well of a transwell plate. The well without DMGF was used as a control group of experiment. DMEM cell culture medium was added into the upper and lower wells, and the lower well was further added fetal bovine serum, and then maintained in the incubator under 37° C. In other embodiment, B16F10 cell suspension was allowed to incubate in the 4 or 6 upper wells of transwell plate to comduct second repeat or duplicate tests, which is not for limited sense of present invention.

The bottom side of the upper well, which is between the upper well and the lower well, includes a transparent film. The present example applied 8 μm (coated with matrigel) transparent films for tumor metastasis test. B16F10 cells migrated through the membrane of transwell chambers from upper layer to the lower layer with DMEM growth medium containing FBS. Generally speaking, B16F10 cells were allowed to migrate toward the lower layer for 4 hours incubation. The cells on the top of the filter were removed with a cotton swab and migrated cells on the underside were fixed with methanol, stained with 50 μg/ml propidium iodide and counted with a fluorescence microscope.

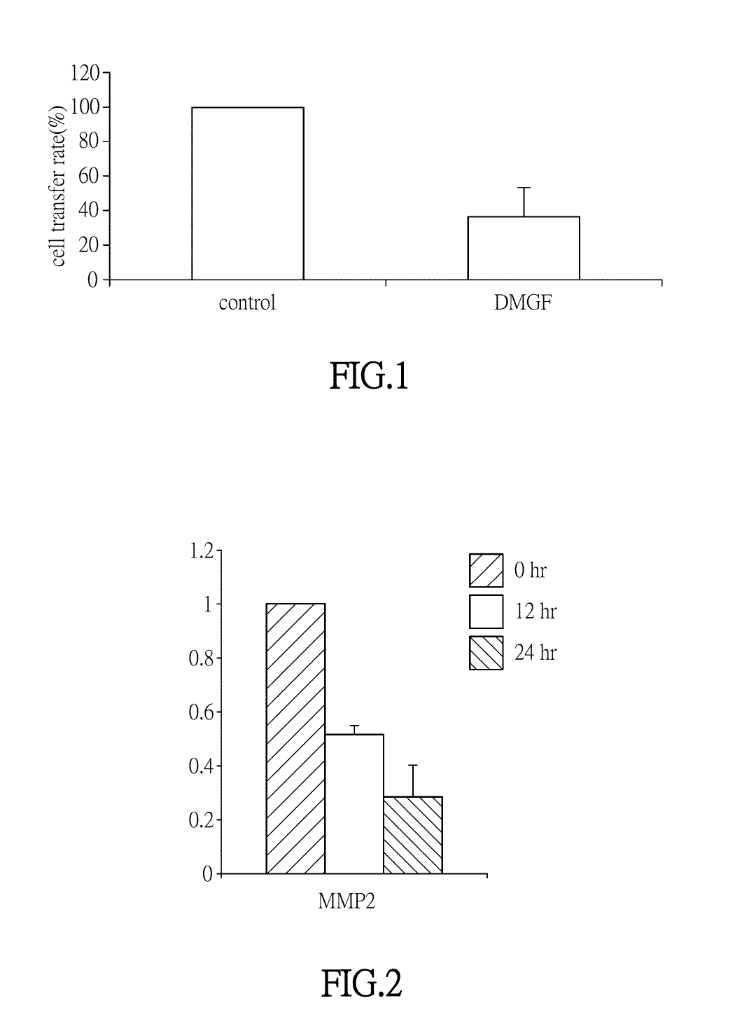

FIG. 1 shows data figure for calculating the B16F10 cells migrating through transwell plate to the lower well in example 2. With reference to FIG. 1, transfer rate of the control group of the experiment was defined 100% to calculate the transfer rate of the B16F10 cells treated with DMGF (marked biflavonoid compound DMGF). FIG. 1 showed about 65% reduction in the capacity of DMGF treated B16F10 cells migrate through the membrane of transwell chambers compared to the group without DMGF, which proved that the pharmaceutical composition is really effective in inhibiting tumor metastasis of tumor cells, especially melanoma cells.

In addition, since the transparent film of the transwell plate was coated with matrigel similar to extracellular matrix, tumor cell may migrate to the lower well by degrading the extracellular matrix with protease secreted from melanoma tumor cells. That is, pharmaceutical composition containing biflavonoid compound DMGF includes the ability of inhibiting tumor cells from degrading extracellular matrix, thus inhibiting tumor metastasis.

### EXAMPLE 3

### Assay for Matrix Metalloproteases-2 (MMP2) Inhibition by DMGF

Culturing condition of B16F10 cells with tissue culture techniques is the same as example 2. B16F10 cell suspension was incubated in at least three plates, two of which were added biflavonoid DMGF (final concentration 1 μg/ml) and set back to 37° C. incubator and incubating for 12 and 24 hours, respectively. The culture medium and additional compound were sucked, and then the plate was washed with PBS. B16F10 cells were collected with trypsin-EDTA. And the third well was processes without DMGF, colleting B16F10 cells according to the above-mentioned method.

Then total B16F10 cellular RNA was extracted with TRIzol (Invitrogen Life Technologies, Carlsbad, Calif., USA) and reverse-transcribed into cDNA using the extracted RNA template. The cDNA was further a template for real-time polymerase chain reaction (Real-time PCR). The forward primer and the reverse primer of MMP2 were 5′-CAC CTG GTT TCA CCC TTT CTG-3′ and 5′-AAC GAG CGA AGG GCA TAC AA-3′, repectively, thus quantified the MMP2 expression level in the above-mentioned time period of control group and experiment group of the present example.

In detail, reverse-transcription PCR was treated with SuperScript First-Strand system (Invitrogen Life Technologies). Real-time PCR was applied a system using fluorescent dye as a detection method (Applied Biosystems, Carlsbad, Calif., USA). The reagent (with total volume 25 μl) for real-time PCR were as follows: 1 μl cDNA, 0.25 μl forward primer of MMP2, 0.25 μl reverse primer of MMP2, 11 μl DDW, and 12.5 μl 12X realQ PCR master mix (with 10 mM MgCl2, Green DNA dye). Real-time PCR consists of a 95° C. denaturation step for 10 min and followed by 40 cycles of 15 s at 95° C., 1 min at 60° C. to proliferate the DNA fragment if MMP2.

The experiment method of extracting RNA with TRIzol, cDNA PCR and real-time PCR are well-understood by the person having ordinary skill in the art, and are not repeated here.

FIG. 2 shows data figure of MMP2 expression level in example 3. With reference to FIG. 2, “0 hour” group is MMP2 RNA expression level of B16F10 cell treated without DMGF, “12 hours” group and “24 hours” group are MMP2 RNA expression level of B16F10 cell treated with DMGF for 12 and 24 hours, respectively. While the MMP2 expression level of “0 hour” group is defined as 1 times, the MMP2 expression level of “12 hours” group and “24 hours” group are both calculated according to the “0 hour” group. As the result shown in FIG. 2, the MMP2 expression levels of B16F10 cell are 0.5 times and 0.3 times, respectively. Therefore, according to example 3, pharmaceutical composition containing DMGF significantly inhibited the MMP-2 expression in tumor cells, especially melanoma cells.

MMP2 belonging to a protease has the ability of degrading extracellular matrix. From example 3, DMGF inhibits extracellular matrix degradation ability of tumor cells by inhibiting MMP2 expression; otherwise, DMGF is capable of inhibiting tumor metastasis by inhibiting protease of tumor cells.

### EXAMPLE 4

### Inhibiting the Polymerization of Actin Filaments with DMGF

Tissue culture technology for culturing SVEC4-10 mouse vascular endothelial cells can be referred to the culturing method of B16F10 in example 2, and is not repeated here. SVEC4-10 cell suspension (3×10⁴) were incubated in at least two plates and treated with 16 ng/ml VEGF (Upstate Inc., Lake Placid, N.Y., USA) medium in the two 96-well plate, and one of the plate was further added with 1 μg/ml DMGF as an experiment group. After adding the culture medium and the above-mentioned compound, SVEC4-10 cells were incubated in 37° C. incubator for 3 to 4 hours and then dyed with diamidino-phenyl-indole (DAPI) and Phallotoxin, respectively. Diamidino-phenyl-indole can be combined with double-stranded DNA to label the position of nucleus. Phallotoxin capable of combining with fibrous actin is often used as the dye for observing the polymerization of fibrous actin. After dyeing, the polymerization of fibrous actin was observed under 40 magnification microscopic lens.

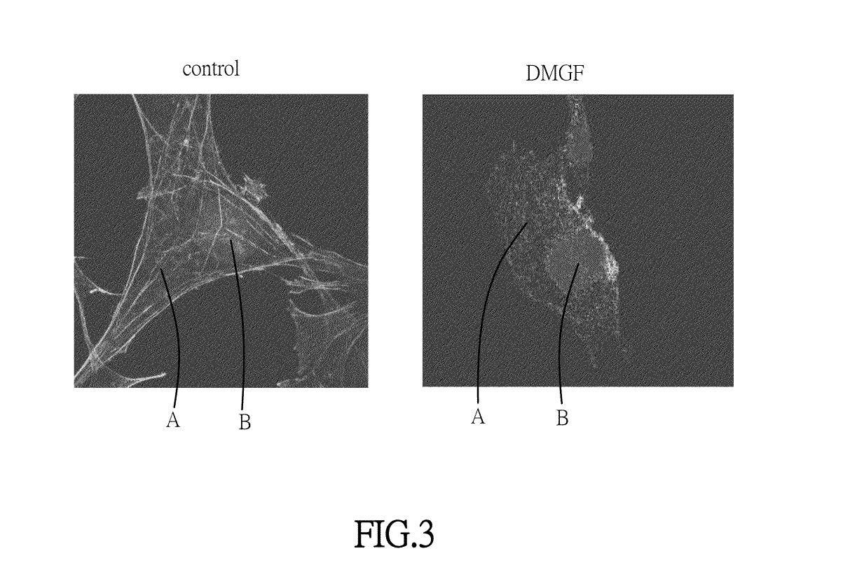

FIG. 3 shows the result of fibrous actin polymerization of SVEC4-10 in example 4. “A” (filaments or spot) in FIG.3 marks the structure of fibrous actin, and “B” (circle) in FIG. 3 marks the position of nucleus. As shown in FIG. 3, in the control group, the fibrous actin of A-marked position apparently shows that fibrous actins twisted with each other to form microfilament helix; however, the fibrous actin of A-marked position in the DMGF group apparently shows multiple dots without polymerization of fibrous actin like A-marked position in the control group. Polymerization of fibrous actin is correlated with cell migration in which polymerization of fibrous actin happens. Cell migration is the early step of tumor metastasis. Hence, according to the result of example 4, pharmaceutical composition with DMGF indeed has the efficacy of inhibiting fibrous actin polymerization, thus inhibiting tumor metastasis.

### EXAMPLE 5

### Anti-Metastatic Activity of DMGF

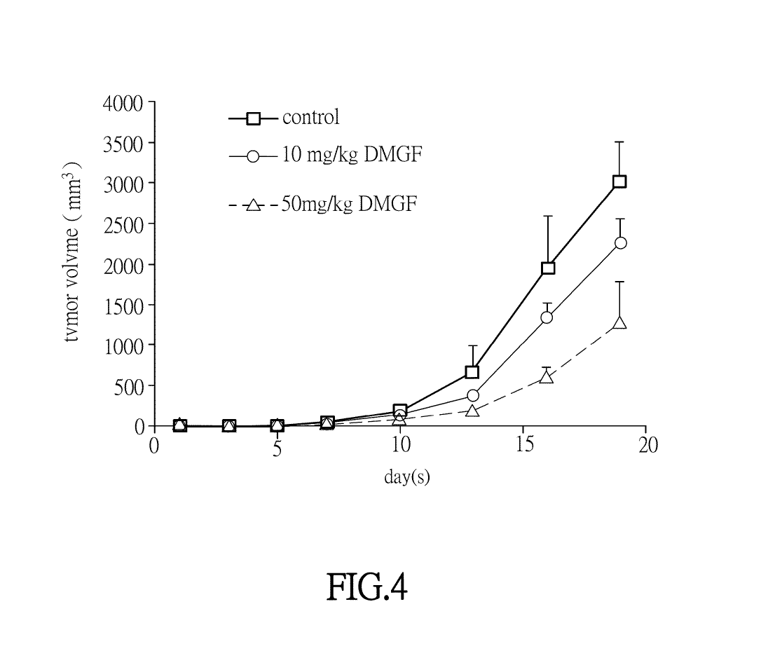

At least 3 8-week-old C57BL/6 mice (female) were i.v. injected with 1×10⁶ B16F10 cells. Mice bearing tumors with more than 30 mm³ were injected with PBS, 10 mg/kg DMGF and 50 mg/kg DMGF once every 2 to 3 days. The result is shown in FIG. 4. FIG. 4 shows the result of the tumor volume of each mouse in example 5. With reference to FIG. 4, tumor volume of mouse treated with DMGF is significantly smaller than that of mouse treated only with PBS (without DMGF). That is, with reference to the result of example 5, pharmaceutical composition with DMGF indeed inhibits tumor cell migration, thus inhibiting tumor metastasis.

According to example 5, acceptable DMGF dosage for mice with C57BL/6 tumor approximately ranges between 10 and 50 mg/kg per day. The dosage for mice is taken into the human effective dosage (HED) equation “HED (mg/kg)=mice dosage (mg/kg)×mice Km factor÷human Km factor”. The mice dosage of the present invention ranges between 10 mg/kg and 50 mg/kg, the mice Km factor is 3, and the human Km factor, thus summing that the HED range is between 0.8 mg/kg and 4 mg/kg.

### EXPERIMENT 6

### Anti-Inflammation of DMGF

1×10⁶ mouse splenocyte suspension was seeded in 24-well culture plate supplemented with 1 μg/ml lipopolysaccharide (LPS) for inducing the immune cells of mouse splenocyte secreting TNF-α, and 0, 0.3125, 0.625, 1.25 μg/ml DMGF to each group. The mouse splenocyte were further incubated in 37° C. incubator for 72 hours. The culture medium was collected and tested for TNF-α concentration by ELISA kit for detecting TNF-α.

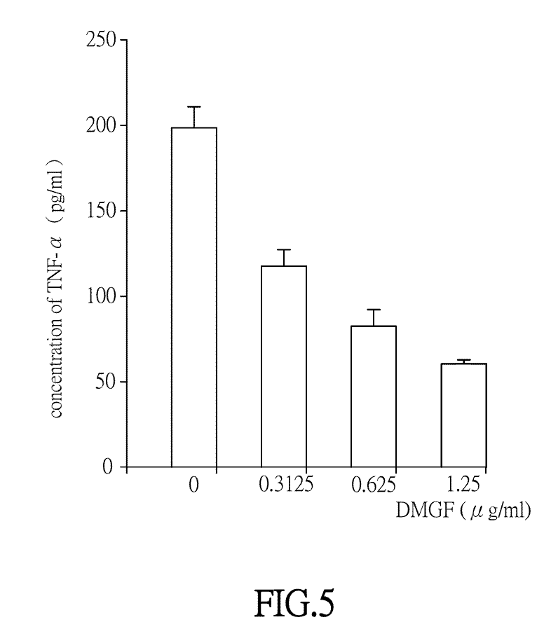

FIG. 5 shows the result of TNF-α secreted by mouse splenocyte in example 6. As shown in FIG. 5, the mouse splenocyte group without DMGF treatment has TNF-α concentration up to 200 pg/ml. With the use of increased DMGF dose level, the lower the TNF-α concentration. For example, mouse splenocyte treated with 1.25 μg/ml DMGF secreted TNF-α concentration fell to 60 pg/ml. With reference to the result of example 6, pharmaceutical composition with DMGF has the ability to inhibit immune cells secreting TNF-α to inhibit inflammation. Since inflammation is related to improving angiogenesis and invasion, migration or other pathological effects of tumor cells, pharmaceutical composition with DMGF has the ability to inhibit inflammation, thus inhibiting tumor metastasis.

In sum, the pharmaceutical composition used for inhibiting tumor metastasis, including biflavonoid compound DMGF capable of inhibiting tumor cells degrading the extracellular matrix coated on the transwell plate. In view of RNA level, DMGF can effectively inhibit RNA expression of MMP2. MMP2 is a kind of protease. Hence, inhibiting MMP2 (or protease) expression may further inhibit the extracellular matrix degradation ability of tumor cell, thus inhibiting the tumor metastasis.

Otherwise, DMGF can effectively inhibit fibrous actin polymerization and metastasis. In addition, DMGF is capable of inhibiting immune cells secreting TNF-α to suppress inflammation, thus preventing tumor cells from invasion and metastasis.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A pharmaceutical composition for inhibiting tumor metastasis which comprises 7,7″-Dimethoxyagastisflavone and a pharmaceutically acceptable carrier.

2. The pharmaceutical composition according to claim 1, wherein the tumor includes melanoma, lung cancer or colorectal cancer.

3. The pharmaceutical composition according to claim 1, wherein the 7,7″-Dimethoxyagastisflavone inhibits the extracellular matrix degradation ability of tumor cells to inhibit tumor metastasis.

4. The pharmaceutical composition according to claim 3, wherein the 7,7″-Dimethoxyagastisflavone inhibits the ability of tumor cells degrading extracellular matrix by inhibiting the expression of matrix metalloproteases-2.

5. The pharmaceutical composition according to claim 1, wherein the 7,7″-Dimethoxyagastisflavone inhibits tumor metastasis by inhibiting protease of tumor cells.

6. The pharmaceutical composition according to claim 1, wherein the 7,7″-Dimethoxyagastisflavone inhibits tumor metastasis by inhibiting fibrous actin.

7. The pharmaceutical composition according to claim 1, wherein the 7,7″-Dimethoxyagastisflavone inhibits tumor metastasis by inhibiting immune cells secreting TNF-α.

8. The pharmaceutical composition according to claim 1, wherein the dosage of 7,7″-Dimethoxyagastisflavone ranges between 0.8 mg/kg per day and 4 mg/kg per day.

9. A use of a 7,7″-Dimethoxyagastisflavone applied to inhibit tumor metastasis.

10. The use according to claim 9, wherein the tumor includes melanoma, lung cancer or colorectal cancer.

11. The use according to claim 9, wherein the 7,7″-Dimethoxyagastisflavone inhibits the extracellular matrix degradation ability of tumor cells to inhibit tumor metastasis.

12. The use according to claim 11, wherein the 7,7″-Dimethoxyagastisflavone inhibits the ability of tumor cells for degrading extracellular matrix by inhibiting the expression of matrix metalloproteases-2.

13. The use according to claim 9, wherein the 7,7″-Dimethoxyagastisflavone inhibits tumor metastasis by inhibiting protease of tumor cells.

14. The use according to claim 9, wherein the 7,7″-Dimethoxyagastisflavone inhibits tumor metastasis by inhibiting fibrous actin.

15. The use according to claim 9, wherein the 7,7″-Dimethoxyagastisflavone inhibits tumor metastasis by inhibiting immune cells secreting TNF-α.

16. The use according to claim 9, wherein the dosage of 7,7″-Dimethoxyagastisflavone ranges between 0.8 mg/kg per day and 4 mg/kg per day.
